(19) United States
(12) Patent Application Publication
(10) Pub. No.: US 2006/0089661 A1
(43) Pub. Date: Apr. 27, 2006

(54) Omni-actuable hand-held surgical instruments

(76) Inventors: Brian C Dodge, Durham, NC (US); Ronald F Overaker, Durham, NC (US); Cynthia A Toth, Durham, NC (US)

Correspondence Address: THOMPSON COBURN, LLP, ONE US BANK PLAZA, SUITE 3500, ST LOUIS, MO, 63101, US

(21) Appl. No.: 10/512,899

(22) PCT Filed: May 5, 2003
(86) PCT No.: PCT/US03/13922

§ 371 Date: Oct. 11, 2005

Related U.S. Application Data
(60) Provisional application No. 60/377,603, filed on May 6, 2002.

(51) Int. Cl. A61F 9/00

(52) U.S. Cl. 606/166

(57) ABSTRACT

Hand-held instruments which may be employed to actuate a distally mounted tool by application of radial compressive force about the instrument circumference (i.e., omni-actuatable). In preferred forms, hand-held instruments have a handle and a slide ring assembly attached to the handle which defines a generally V-shaped circumferential channel. The slide ring assembly will have at least one slide ring mounted for longitudinal movements relative to the handle. An actuator member is operatively engaged with the slide ring assembly for moving the slide ring longitudinally relative to the handle in response to a force applied against the actuator member. In preferred embodiments, the actuator member is tubular and includes at least one actuation finger, and more preferably a circumferentially disposed plurality of actuation fingers. The actuator member includes a tubular proximal end section thereof sleeved over the handle so as to be located proximally of the slide ring assembly. The actuation fingers thus extend distally from the tubular proximal end section to (and preferably distally beyond) the slide ring assembly. The actuation fingers are provided a radially inwardly protruding actuator nib which is seated in the generally V-shaped channel of said slide assembly. Thus, in response to a compressive force applied against the actuator fingers, the nib thereof will be depressed radially within the channel thereby causing the slide ring to move longitudinally which in turn, coacts operatively with the distally mounted tool.

### FIELD OF THE INVENTION

The present invention relates generally to hand-held instruments and tools. In preferred forms, the present invention relates to hand-held surgical instruments, especially those usefully employed for ophthalmic surgical procedures.

### BACKGROUND OF THE INVENTION

Ophthalmic surgical procedures require the use of miniaturized instruments such as, for example, forceps, scissors and the like in order to allow the surgeon to operate on and within a patient's eye. One well known instrument that is used for such ophthalmic surgical procedures is the so-called Sutherland-type instrument commercially available from Grieshaber & Co., Inc. The Sutherland-type instrument has a pen-like handle and uses a lever as an actuator for actuating mechanically operable spring-loaded tools, such as forceps, scissors, knives and the like which are threaded or otherwise operably connected to the distal end of the handle.

Recently, improvements to such Sutherland-type instruments have been proposed in U.S. Pat. No. 5,634,918 (the entire content of which is expressly incorporated hereinto by reference). In general, the improvements proposed by the '918 Patent include providing a circumferentially arranged series of lever-like triggers which are pivotal in response to a radial force being applied thereto. Radially inward and outward pivotal movements of one trigger will, in turn, be converted respectively into rightward and leftward translation of the trigger retainer and is accompanied by like simultaneous movement of all the other triggers. Thus, any working tool attached operable to the trigger retainer will likewise translate rightward and leftward therewith.

A further variation of the Sutherland-type instrument that has been employed in the past is to provide an opposed pair of arced spring elements which protrude outwardly from the handle. A pair of relatively narrow slats extends from a proximal end of the handle distally so that each is positioned over a respective one of the protruding spring elements. Application of pressure onto the tabs will thus depress the arced springs thereby operating a working tool at a distal end of the instrument. Such a modification does not, however, allow for full 360° operation around the circumference of the handle.

Further improvements to Sutherland-type instruments have been proposed in copending U.S. patent application Ser. No. 09/549,469 filed on Apr. 14, 2000 (“the '469 application”) and Ser. No. 09/837,555 filed on Apr. 19, 2001 (“the '555 application”), the entire content of each prior filed patent application being expressly incorporated hereinto by reference. More specifically, the '469 application discloses omni-actuatable hand-held surgical instruments in which a radially flexible actuator band is seated in a generally V-shaped circumferential channel defined between a pair of rings, at least one of which is longitudinally moveable. In response to a radially compressive force. The actuator band will therefore be flexed radially inwardly so as to longitudinally move at least one of the slide rings, and hence a distally mounted tool operatively connected thereto.

Surgical instruments are also proposed to be omni-actuatable according to the '555 application. In preferred forms, the invention of the '555 application is embodied in hand-held instruments having a handle which includes an actuator assembly for actuating a tool, wherein the actuator assembly includes a plurality of circumferentially spaced-apart generally L-shaped actuator levers defining respective arcuate bearing surfaces along exterior edge regions thereof, and a retaining ring which circumferentially bounds the actuator levers around the exterior edge regions thereof. The retaining ring most preferably defines an interior stationary arcuate guide surface in conformable mated relationship to the bearing surfaces of said actuator levers. Upon application of a radially inwardly directed force against the proximally extending arms of the actuator levers, the actuator levers are caused to move collectively toward the holder 14 (that is, to be moved to a position closer to the central axis of the instrument) which movement, in turn, causes the generally radially inwardly extending legs of the actuator levers to be thrown in a distal direction, thereby operating the attached surgical tool.

### SUMMARY OF THE INVENTION

The present invention is directed to further improvements in surgical instruments of the Sutherland-type. In this regard, the present invention broadly is directed to hand-held instruments which may be employed to actuate a distally mounted tool by application of radial force about the instrument circumference (i.e., omni-actuatable). In preferred forms, the present invention is embodied in hand-held instruments having a handle which includes an actuator assembly for actuating a tool, wherein the actuator assembly has a generally V-shaped circumferential channel in which an actuator band is seated. A slide ring assembly is provided which defines the generally V-shaped circumferential channel and has at least one slide ring mounted for longitudinal movements relative to the handle.

The actuator is most preferably in the form of a tubular member having a series of lengthwise, circumferentially spaced-apart slits in one end which in turn define a plurality of circumferentially spaced-apart flexible actuation fingers. The actuation fingers are provided with radially inwardly directed actuator nibs which are seated in the generally V-shaped channel of the slide assembly. Thus, compressive force applied against the actuation fingers will cause the nibs thereof to be depressed within the V-shaped channel thereby causing a slide ring of the assembly to be longitudinally displaced in a distal direction. Such longitudinal displacement of the slide ring will thereby operatively cause a distal tool of the instrument to be operated.

These and other aspects and advantages will become more apparent after careful consideration is given to the following detailed description of the preferred exemplary embodiments thereof.

### BRIEF DESCRIPTION OF THE ACCOMPANYING DRAWINGS

Reference will hereinafter be made to the accompanying drawings, wherein like reference numerals throughout the various FIGURES denote like structural elements, and wherein;

FIG. 1 is a perspective view of a preferred embodiment of a hand-held surgical instrument according to the present invention in use;

FIG. 2 is an enlarged exploded perspective view instrument showing the actuator member removed physically from the surgical instrument handle; and

FIGS. 3A and 3B are each longitudinal views with the actuator member cross-sectioned showing the operation of the surgical instrument in accordance with the present invention.

### DETAILED DESCRIPTION OF THE INVENTION

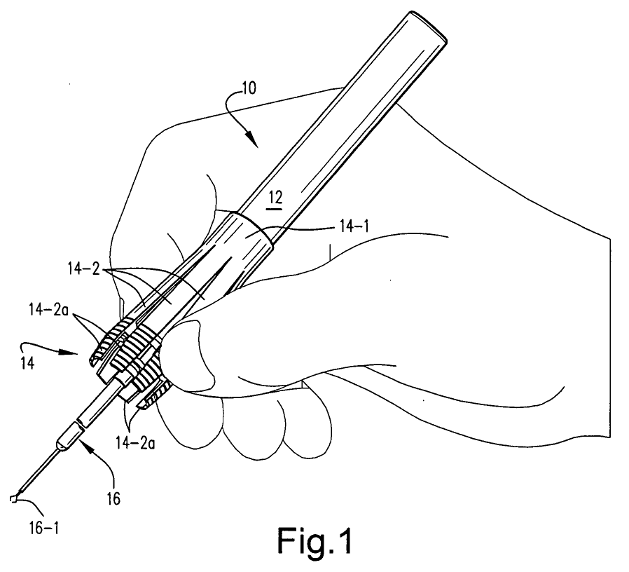

Accompanying FIG. 1 depicts one exemplary embodiment of a hand-held surgical instrument 10 according to the present invention. In this regard, the surgical instrument 10 includes an elongated handle 12 sized and configured to allow the instrument 10 to be handled manually by a surgeon during surgical procedures. The handle 12 includes a manually operated actuator member 14 which serves to actuate a tool 16 operatively attached to, and extending from, the distal end of the handle 12. The tool 16, for example, may be a miniature forceps 16-1 positioned at the distal-most end thereof which open and close in response to actuation of the actuator member 16 in a manner that will be described in greater detail below.

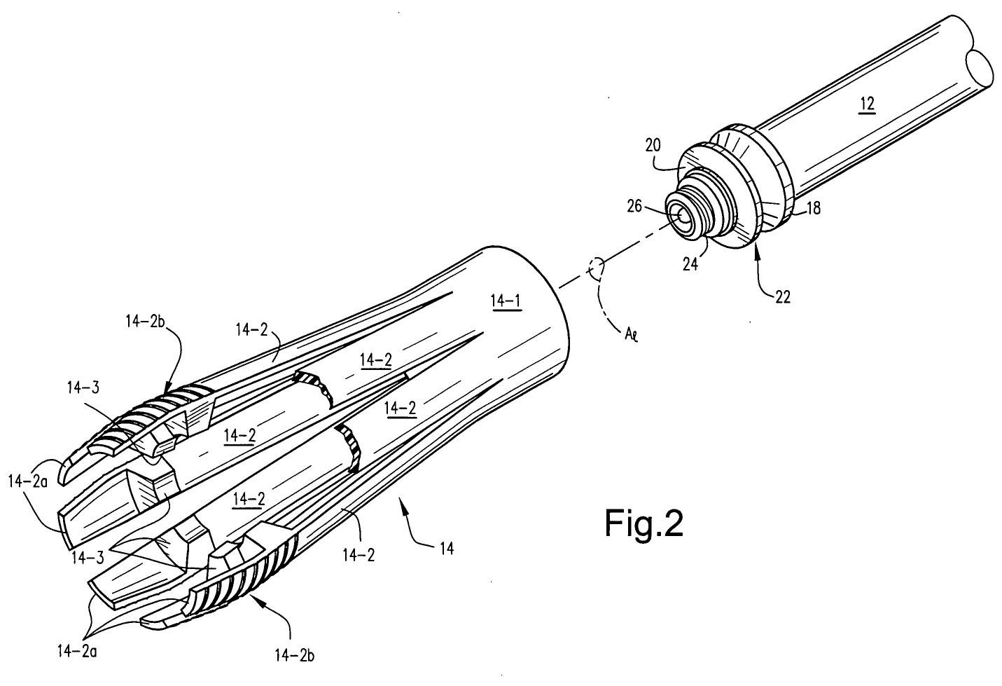

As is perhaps more clearly shown in FIG. 2, the distal end of the handle 12 includes a fixed-position annular actuation flange 18 and an annular slide ring 20 which is capable of slideable reciprocal rectilinear movements along the elongate axis (Aₗ in FIG. 2) of the instrument 10. The actuation flange 18 and the slide ring 20 define opposed radially inwardly converging respective ramp surfaces which thereby define a generally V-shaped channel 22. The handle 12 terminates distally in a threaded nipple 24 for removeably threadably receiving a proximal end of the surgical tool 16. Removable connection means between the handle 12 and the surgical tool 16 other than threaded connection as shown may be employed in accordance with the present invention. For example, bayonet-type connections, frictional detent-type connections, magnetic connections and other like removable structural connections may be employed, if desired. An actuator pin 26 is positioned within the coupling 24 and is connected operatively to the slide ring 20 in the manner described more fully in the above-cited '469 application. Thus, longitudinal distal and proximal displacements of the slide ring 26 relative to the elongate axis Aₗ will responsively cause the pin 26 to coaxially extend and retract thereby operating the tool 16.

The actuator member 14 is a generally tubular structure that is sized so as to be sleeved over the handle 12. Most preferably, the actuator member 14 is a longitudinally slit, one-piece (i.e., unitary) body of a diameter that allows it to be sleeved over the handle 12. The actuator member 14 will thus include a proximal (non-slit) tubular end section 14-1 positioned at a location proximally of the flange 18 and slide ring 20, and a plurality of circumferentially disposed actuator fingers 14-2 which extend distally from the tubular end section 14-1. In the preferred embodiment depicted in the accompanying drawing FIGURES, a total of eight (8) actuator fingers 14-2 are provided. However, greater or lesser number of actuator fingers 14-2 could be provided as may be desired by the manufacture and/or end user.

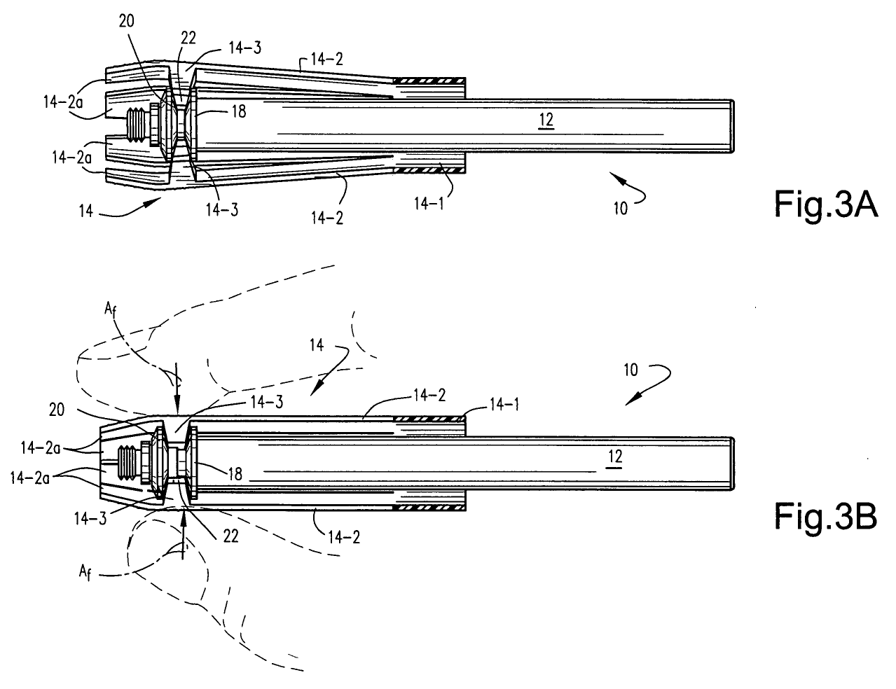

The actuator fingers 14-2 are provided with radially inwardly protruding actuator nibs 14-3 which, as is perhaps shown more clearly in FIGS. 3A and 3B, are in use seated in the V-shaped channel 22. The cross-sectional shape of the actuator nibs 14-3 is in the general form of a wedge, however, the surfaces thereof which contact the ramp surfaces of the actuation flange 18 and the slide ring 20 do not necessarily need to be planar. Thus, like the ramp surfaces, the surfaces of the actuator nibs 14-3 may be convexly curved so as to promote essentially tangential contact between the nibs 14-3 and the ramp surfaces of the actuation flange 18 and slide ring 20.

The actuation fingers 14-2 are most preferably of sufficient length that the distal terminal ends 14-2a thereof extend distally beyond the slide ring 20 (and hence also distally beyond the nibs 14-3 thereof). Furthermore, the actuator fingers 14-2 are most preferably formed so as to inwardly converge in a distal direction to thereby enhance grasping and manipulation of the instrument 10 in the surgeon's hand. In addition, an exterior portion of the distal ends 14-2a of the actuator fingers 14-2 may be provided with serrations 14-2b so as to promote a more rough feel to the attending surgeon. Other means may also be employed in order to improve the tactile sensation and/or feel of the actuation levers 14-2, such as, for example coating at least the exposed exterior surfaces thereof with a friction material (e.g., an elastomeric material) or the like.

The operation of the actuator member 14 employed in the hand-held surgical device 10 in accordance with the present invention is depicted generally in accompanying FIGS. 3A and 3B. In this regard, it will be understood that a proximal end of the tool 16 (see FIG. 1) will be threadably and removeably connected to the threaded nipple 24 coaxially extending distally from the handle 12. As noted above, virtually any other means may be employed so as to achieve removable interconnection between the handle 12 and the tool 16, if desired. Although not shown, the tool 16 will conventionally have an actuator rod which extends through the nipple 24 proximally into operative engagement with the pin 26. Moreover, the tool 16 is conventionally provided with a spring element which urges the actuation rod thereof into contact with the activation pin 26. Thus, the force of the tool's spring element will cause the actuation pin 26 to be displaced proximally so as to cause the slide ring 20 operatively attached thereto to assume a “normal” or rest condition as shown in FIG. 3A.

Upon application of a radially inwardly directed force against the exterior surface of at least one, and more preferably at least on opposed pair, of the actuation fingers 14-2 (as noted by the arrows A_{f} in FIG. 3A), the associated actuator nibs 14-3 will be caused to be radially depressed within the generally V-shaped channel 22 defined between the actuation flange 18 and the slide ring 20. Since the cross-section shape of the nibs 14-3 is generally wedge-shaped such that they have a greater cross-sectional thickness (as measured along the axis Aₗ) near the fingers 14-2 and decrease in radial thickness near the terminal ends thereof, the slide ring 20 will responsively be displaced longitudinally from its rest position shown in FIG. 3A and into its operative position shown in FIG. 3B. This longitudinal movement will, in turn, cause the pin 26 (see FIG. 2) to extend distally thereby operating the tool 16. When the force A_{f} is released, the spring associated with the tool 16 will drive the pin 26 rearwardly, thereby returning the slide ring 20 and the actuation fingers 14-2 to their respective rest positions as shown in FIG. 3A.

The length of the actuation fingers 14-3 relative to the proximal tubular end section 14-1 will determine the amount of spring action (“springiness”) which the actuation fingers 14-3 exhibit in use. Thus, greater or lesser lengths of the actuation fingers 14-3 may be provided by those skilled in the art so as to adjust their respective spring action. Alternatively or additionally, the material from which the actuator member 14 (and hence the actuation fingers 14-3) is made can also be selectively chosen so as to provide an actuator member with desired spring characteristics. Suffice it to say, those skilled in the art will recognize various techniques that may be employed in order to “engineer” the desired spring characteristics of the actuation fingers 14-3.

Since the actuation member 14 is most preferably simply removeably sleeved over the handle 12 (and thus is not necessarily permanently fixed thereto) it is contemplated that a number of actuation members 14 could be provided in kit form such that the individual actuation members 14 differ from one another in a variety of characteristics, for example, differing circumferential size (i.e., greater and/or lesser overall diameters), and/or differing longitudinal lengths, and/or differing spring characteristics, to name just a few. Such different actuation members 14 may also be color-coded so as to visually identify their individual characteristics. In this manner, a surgeon could personally select a desired actuation member 14 to suit a particular tool and/or surgical procedure and could change from one actuator to another easily during the same operation.

A single slide ring 20 has also been depicted which forms a single generally V-shaped channel so as to receive the actuator nibs 14-3 therein and thus represents a presently preferred form of the invention. However, more than a single such slide ring may be provided without departing from the scope of the present invention, in which case the actuator fingers 14-2 are likewise provided with multiple nibs 14-3 seated in respective V-shaped channels thereof.

Although the actuator 14 in accordance with the present invention has been disclosed as being used in combination with a V-shaped actuation channel defined between the flange 18 and slide ring 20, it will be appreciated by those skilled in the art that such a combination represents a particularly preferred embodiment of the present invention. The actuator 14 of the present invention could, however, be employed in combination with other structural systems. Suffice it to say that those skilled in the art may recognize other potential structural combinations and/or uses that may employ the actuator 14 of the present invention.

Therefore, while the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A hand-held instrument comprising:
a handle;
a slide ring assembly attached to the handle which defines a generally V-shaped circumferential channel and has at least one slide ring mounted for longitudinal movements relative to the handle; and
an actuator member operatively engaged with the slide ring assembly for moving said at least one slide ring longitudinally relative to the handle in response to a force applied against the actuator member, wherein
said actuator member includes at least one actuation finger having an end section thereof located proximally of the slide ring assembly and extending distally from said end section to said slide ring assembly, and wherein
said at least one actuation finger includes an actuator nib seated in said generally V-shaped channel of said slide assembly.

2. The instrument of claim 1, wherein said actuator member is generally tubular in configuration and includes a circumferentially arranged plurality of said actuation fingers.

3. The instrument of claim 2, wherein said actuator member includes a tubular proximal end section which is sleeved over said handle.

4. The instrument of claim 1, wherein said at least one actuator finger includes a terminal end section which extends distally beyond said slide ring assembly.

5. The instrument of claim 4, wherein said terminal end section is formed so as to converge distally inwardly.

6. The instrument of claim 4, wherein an exterior surface of said terminal end section includes means for imparting tactile sensation to a user.

7. The instrument of claim 6, wherein said means include serrations.

8. The instrument of claim 1, wherein said actuator nib of said at least one actuation finger has a generally wedge-shaped cross-section.

9. A hand-held instrument comprising:
a handle;
a slide ring assembly attached to the handle which defines a generally V-shaped circumferential channel and has at least one slide ring mounted for longitudinal movements relative to the handle; and
a tubular actuator member sleeved over said handle and operatively engaged with the slide ring assembly for moving said at least one slide ring longitudinally relative to the handle in response to a force applied against the actuator member, wherein
said actuator member includes a tubular proximal end section which is sleeved over said handle at a location proximally of said slide ring assembly, and a plurality of circumferentially disposed actuation fingers extending distally from said tubular end section to said slide ring assembly, and wherein
said each of said actuation fingers includes an actuator nib seated in said generally V-shaped channel of said slide assembly.

10. The instrument of claim 9, wherein said actuator nib of said actuation fingers has a generally wedge-shaped cross-section.

11. The instrument of claim 9, wherein each of said actuator fingers includes a terminal end section which extends distally beyond said slide ring assembly.

12. The instrument of claim 11, wherein said terminal end section is formed so as to converge distally inwardly.

13. The instrument of claim 11, wherein an exterior surface of said terminal end section includes means for imparting tactile sensation to a user.

14. The instrument of claim 13, wherein said means include serrations.

15. An tubular actuator member adapted to being sleeved over a handle of a hand-held instrument, said actuator member including:
a tubular proximal end section which is sized and configured to be sleeved over the handle of the hand-held instrument; and
a plurality of circumferentially disposed actuation fingers extending distally from said tubular end section; wherein
each of said actuation fingers includes a radially inwardly protruding actuator nib.

16. The tubular actuator member of claim 15, wherein said actuator nib of said actuation fingers has a generally wedge-shaped cross-section.

17. The tubular actuator of claim 9, wherein each of said actuator fingers includes a terminal end section which extends distally beyond said nib.

18. The tubular actuator of claim 17, wherein said terminal end section is formed so as to converge distally inwardly.

19. The tubular actuator of claim 17, wherein an exterior surface of said terminal end section includes means for imparting tactile sensation to a user.

20. The tubular actuator of claim 19, wherein said means include serrations.

21. An actuator for a surgical instrument comprising a generally tubular body having a series of circumferentially spaced-apart slits formed in one end thereof which establish a plurality of longitudinally extending flexible actuation fingers.

22. The actuator of claim 21, wherein said actuation fingers include radially inwardly protruding actuator nibs.

23. The actuator of claim 22, wherein said actuator nibs of said actuation fingers have generally wedge-shaped cross-sections.

24. The actuator of claim 22, wherein said actuator fingers include terminal end sections which extend distally beyond said nibs.

25. The actuator of claim 24, wherein said terminal end sections converge distally inwardly.

26. The actuator of claim 25, wherein exterior surfaces of said terminal end sections include means for imparting tactile sensation to a user.

27. The actuator of claim 26, wherein said means include serrations.

28. A surgical instrument comprising a proximal handle, a distal tool, and an actuator as in claim 21 sleeved over said handle for actuating said tool.

29. A kit which comprises a plurality of tubular actuator members as in claim 21, and wherein at least one of said tubular actuator members is different in at least one of size, shape, color and spring action as compared to another of said tubular actuator members.
